(19) United States
(12) Patent Application Publication
(10) Pub. No.: US 2005/0084509 A1
(43) Pub. Date: Apr. 21, 2005

(54) Use of citric acid and low concentrations of alpha-hydroxy acids for superficial skin treatment

(76) Inventor: Eric F. Bernstein, Gladwyne, PA (US)

Correspondence Address: Licata & Tyrrell P.C., 66 East Main Street, Marlton, NJ, 08053, US

(21) Appl. No.: 10/689,002

(22) Filed: Oct. 20, 2003

(51) Int. Cl. A61K 7/04; A61K 31/19; A61N 1/00

(52) U.S. Cl. 424/401; 514/557; 514/574; 607/88

(57) ABSTRACT

Methods for improving photoaging, acne, acne scarring and various types of sun damage by topically applying a solution containing citric acid or a low concentration alpha-hydroxy acid for home use are provided.

### BACKGROUND OF THE INVENTION

The effects of ultraviolet radiation from exposure to the sun on human skin are a growing concern for today's longer-lived population. The majority of changes associated with an age appearance result from chronic sun damage. The effects of alpha-hydroxy acids on sun damaged skin are well known. These agents have been shown to reduce the signs of photoaging. In addition, alpha-hydroxy acids have been used in numerous preparations to treat acne. Alpha-hydroxy acids not only treat marks from acne but are also quite effective at treating comedonal and pustular acne. The main alpha-hydroxy acids used in clinical practice are glycolic and lactic acids. Glycolic and lactic acids were selected because they are quite easily produced and are the two smallest molecules in the alpha-hydroxy acid family. Being the smallest molecules confers the advantage of penetrating the skin more easily than larger molecules. Thus, it was believed that glycolic and lactic acids would exert the most beneficial effect in treating photodamage and acne.

Although alpha-hydroxy acids are widely used throughout dermatology around the world, very little is known about their mechanisms of action. Alpha-hydroxy acids are participants in a number of biological pathways. Included in these are the Krebs cycle also known as the citric acid cycle. Lactic acid, another alpha-hydroxy acid, is a byproduct of metabolic pathways in the body as well. Thus, these agents may have receptors present on cell surfaces enabling them to exert effects that would be unexpected by their structure. For example, citric acid is a common preservative found in many foods. It is also formed in the citric acid cycle. Citric acid has been shown to be highly effective for treating ichthyosis despite the large size of the molecule, thus making skin penetration a slower process, compared to glycolic acid. Van Scott and Yu demonstrated that alpha-hydroxy acids are quite effective for treating scaling disorders such as lamellar ichthyosis. Citric acid was shown to be effective at removing excess scale, as were many other alpha-hydroxy acids.

It has now been found that solutions containing citric acid are more effective at reducing the signs of photoaging than an equal concentration of glycolic acid at the same pH. In addition side effects are less from citric acid as compared to glycolic acid of the same concentration. Also, lower concentrations of citric acid that can be left on the skin without any adverse sequelae are being used as peeling agents for home use. The advantages of using these agents with home topical applications followed by neutralization is that the patient gets a superficial peel, while also getting the added safety if some of the solution is not neutralized at the end of the peel procedure and is left on the skin. In addition they get the benefits of the mild exothermic reaction during the neutralization process.

### SUMMARY OF THE INVENTION

The present invention provides a new use for citric acid. It has now been found that citric acid can be used as a tropical peeling agent, when applied as a solution at high concentration (generally from 10-50%). Citric acid peels have been shown to perform better than the commonly used glycolic acid peels and provide an added safety margin. Compositions for using citric acid as a home or doctor's office/spa peeling agent are also provided. The present invention also provides peeling agents comprised a system of administering alpha-hydroxy acid, beta-hydroxy acid, and trichloroacetic acid peels commonly used in doctor's offices and spas but in lower concentrations and/or neutralized forms that are safe for home use. These low concentration alpha-hydroxy acids can also be left safely on the skin un-neutralized without damaging the skin.

### DETAILED DESCRIPTION OF THE INVENTION

Dramatic alterations occur in sun exposed skin as compared to normal sun protected skin. These changes include brown spots, wrinkles, fine lines, skin laxity, and enlarged pores. In addition, skin with acne demonstrates clogged pores, pustules, and papules particularly on the cheeks, nose and chin.

Alpha-hydroxy acids have the ability to strip away the stratum corneum thus improving acne and acne prone skin. In addition, alpha-hydroxy acids including citric acid have the ability to improve both the epidermis and dermis of photodamaged skin. Citric acid can produce an improvement of enlarged pores, brown spots, and fine lines and wrinkles. Adding the citric acid in a solution form and then neutralizing it enables one to effect greater changes than is possible using topical treatments alone. A neutralization process is necessary to ensure removal of all free acid from the skin. It has now been found that use of lower concentration of alpha-hydroxy acids, including, but not limited to, citric acid, approximating ten percent provides for a skin peel system useful in the home. Using a low concentration in this skin peel system ensures that if any un-neutralized solution is left upon the skin that no damage will occur. The skin peel system of the present invention thus enables a patient to derive the physical and psychological benefits of receiving the peel procedure, without the risk that would normally be present with use of high-concentration, low-pH, alpha-hydroxy acids in the home. These solutions are of a strength that can be left on the skin without producing problems. The benefits incurred by receiving the peel are the benefits of the citric acid itself, the mild exothermic reaction that occurs following the neutralization process, and the psychological benefits of having an office-type procedure in the home environment.

The steps for receiving a peel are as follows. The patient's skin is wiped with an alcohol, acetone, or other mild de-greasing agent so that the surface skin oils do not prevent the peel from penetrating the skin surface. Subsequently, a peel solution containing citric acid is applied to the skin. This citric acid solution may be in any concentration but is typically a 10 to 50 percent solution.

Other alpha-hydroxy acids may also be used as home peels for application by consumers at concentration of approximately 5% to 15% percent in a solution or at a higher concentration with some of the free acid neutralized. This home peel may be left on the skin without adverse consequences; unlike the stronger peels used in doctor's offices or spas, which are typically 20-70% solutions. The solution is left in place from approximately two to five minutes or until the patient develops a minor pink appearance to the skin. Then it is neutralized using a sodium bicarbonate solution, which produces bubbles. Once the bubbles have resolved the peel procedure is over. Alternatively, a water rinse can be used to neutralize the peeling system.

Thus, the present invention provides unique alternatives to peeling agents which commonly use glycolic and lactic acids at concentrations of 20% to 70%. In one embodiment, a method is provided for improving skin appearance by topically applying a solution comprising citric acid, preferably at a concentration of 20% to 70%. In another embodiment, a method is provided for improving skin appearance by topically applying a solution comprising an alpha-hydroxy acid at a low concentration of from about 5% to about 15%. Either embodiment of the present invention can be used alone or combination with other treatments including, but not limited to light treatments such as laser treatment, application of a second topical solution, cream or lotion, or an abrasive treatment such as micro-dermabrasion.

For purposes of the present invention, “improving skin appearance” it is meant to encompass improvement in the general appearance, texture and photoaging damage as measured by decreases in fine lines, wrinkles, enlarged pores and/or brown spots following treatment with a method of the present invention.

The following nonlimiting examples are provided to further illustrate the present invention.

### EXAMPLES

### Example 1

### Citric Acid is an Effective Peeling Agent for Photodamaged Skin

Twenty subjects were presented for citric acid peels in a study designed to investigate the effect of citric acid peels on photodamaged skin. Subjects were photographed with a high-resolution digital camera using a head mount to hold the subject's head in place in the exact same position for each photograph. Pictures were taken of the full frontal facial view as well as the left and right sides of the face. In addition, cross-polarized photographs were taken of the face prior to treatment using the same set-up as for the digital plain photographs. Also, Primos topographic imaging was performed on subjects prior to, immediately after, and two months after treatment. The Primos image analysis system takes a three-dimensional map of the skin and is able to assess changes and improvements in the skin following dermatologic treatment. The Primos image analysis system finds the exact location of the skin where the initial image was taken and superimposes a subsequent image, comparing them via computer image analysis. Thus an improvement in fine lines, wrinkles, pore size, and roughness may be calculated. Subjects were all photographed, cross-polarized photographed, and imaged with the Primos image analysis system prior to treatment. The skin was cleansed with a solution containing alcohol and propylene glycol. After cleansing, the areas where the peel can concentrate, such as the sides of the nose and lips, were treated with petrolatum to reduce accumulation of peel in these areas. Then, a 50 percent citric acid solution was applied to the face, and left on for from two to five minutes. The patient's face was then sprayed with a dilute solution containing sodium bicarbonate, which produces carbon dioxide bubbles during this acid-neutralization step. When there are no more bubbles coming from the sprayed area, the neutralization is completed. The patient then dries their face and the peel procedure is finished. Patients underwent citric acid peels at one month intervals and then were evaluated with digital photography, cross-polarized photography, and Primos topographic image analysis two months following the last treatment. A statistically significant improvement was noted in fine lines and wrinkles following the peel treatments. These photographs were evaluated by observers blinded as to the treatment condition. Cross-polarized photography demonstrated no dramatic change in redness. However, immediately following a peel, patients had an increase in redness indicative of inflammation in the skin. This inflammatory response produces the remodeling that occurs following citric acid and other alpha-hydroxy acid peels. Finally, Primos topographic image analysis demonstrated an improvement in fine lines, wrinkles, as well as a reduction in pore size in treated individuals.

### Example 2

### Low Concentration, or Partially Neutralized, Alpha-Hydroxy Acids can be used to Administer Peels in the Home

Ten subjects were treated with a 10 percent solution of citric acid self-administered in the home. Ten percent citric acid may be left on most people's skin without neutralization, making this skin treatment/peel safe for home use. Subjects used the citric acid peel at two-week intervals for a total of three treatments. The peels were administered by the subjects, themselves, in the same method that was outlined in example 1. The face is first washed, then cleansed with a solution containing alcohol and propylene glycol. Following this, the peel solution is applied for a two to five minute time period. The peel is then neutralized using a dilute sodium bicarbonate solution that produces bubbles and a mild warm sensation indicative of the exothermic reaction that is taking place. Two months following the third treatment subjects rated their improvement in pigmentation, redness, fine lines, wrinkles, enlarged pores, and sagging skin. Subjects all experienced a statistically significant improvement in their subjective assessment of fine lines, wrinkles, enlarged pores, and brown spots. The subjects all stated that they wish to acquire more for use in the home.

### References Cited

### Publications

Uitto et al., “Cutaneous Aging: Intrinsic versus extrinsic photoaging”, Retinoids Today & Tomorrow. 1995, 40: 2-4.
Bernstein et al., “Connective tissue alterations in photoaged skin and the effects of alpha-hydroxy acids”, J Geriatr Dermatol. 1995, Suppl A (3): 7A-18A.
Bernstein et al., “The effect of photodamage on dermal extracellular matrix”, Clin Dermatol. 1996, 14: 143-151.
Rendon-Pellerano et al., “The use of glycolic acids in the management of xerosis and photoaging”, J Geriatr Dermatol. 1996, Suppl B (4): 12B-16B.
Bernstein et al., “Glycolic acid treatment increases type I collagen mRNA and hyaluronic acid content of human skin”, Dermatol Surg. 2001, 27: 429-433.
Bernstein et al., “A pilot investigation to subjectively measure treatment effect and side-effect profile of non-ablative skin remodeling using a 532 nm, 2 ms pulse-duration laser”, J Cosmetic & Laser Ther. 2001, 3: 137-141.
Bernstein et al., “Enhanced elastin and fibrillin gene expression in chronically photodamaged skin”, J Invest Dermatol. 1994, 103: 182-186.
Bernstein et al., “Chronic sun-exposure alters the collagen of the superficial dermis: Evaluation by Northern analysis, immunohistochemical staining and confocal scanning laser microscopy”, J Am Acad Dermatol. 1996, 34: 209-218.
Bernstein et al., “Chronic sun-exposed alters both the content and distribution of dermal glycosaminoglycans”, Br J. Dermatol. 1996, 135: 255-262.
Bernstein et al., “Citric acid increases viable epidermal thickness and glycosaminoglycan content of sun-damaged skin”, J Dermatol Surg. 1997, 23: 689-694.
Bernstein et al., “Evaluation of sunscreens with various sun protection factors in a new transgenic mouse model of cutaneous photoaging that measures elastin promoter activation”, J Am Acad Dermatol. 1997, 37: 725-729.
Bernstein et al., “Photoaging effects on dermal extracellular matrix”, Dermatol Cosmetic. 1997, June: 124-127.
Uitto et al., “Molecular mechanisms of cutaneous photoaging”, J Invest Dermatol. 1998, 3: 41-44.
Bernstein et al., “Glycolic acid and chemical peels” Atlas of cutaneous aesthetic surgery. Philadelphia: W. B. Saunders Co., pp. 311-327.
Bernstein et al., “Poly hydroxy acids (PHAs): clinical uses for the next generation of hydroxy acids”, Skin Aging. 2001, 9 Suppl (9): 4-11.
Bernstein et al., “Chemical peels”, Semin Cutan Med Surg. 2002, 21(1): 27-45.
Bernstein et al., “Dermal effects of alpha hydroxy acids”, Glycolic Acid Peels. New York: Marcel Dekker, Inc. in press.

1. A method for improving skin appearance comprising topically applying to the skin citric acid in a concentration of 20% to 70%.

2. The method of claim 1 wherein the citric acid is applied in combination with a light treatment.

3. The method of claim 2 wherein the light treatment comprises laser treatment.

4. The method of claim 1 wherein the citric acid is applied in combination with a second topical solution, cream or lotion.

5. The method of claim 1 wherein the citric acid is applied in combination with an abrasive treatment.

6. The method of claim 5 wherein the abrasive treatment is micro-dermabrasion.

7. A method for improving skin appearance comprising topically applying to the skin an alpha-hydroxy acid at a low concentration of 5% to 15% or a partially neutralized alpha-hydroxy acid which is non-irritating to the skin.

8. The method of claim 7 wherein the alpha-hydroxy acid is citric acid.

9. The method of claim 7 wherein the alpha-hydroxy acid is applied in combination with a light treatment.

10. The method of claim 9 wherein the light treatment comprises laser treatment.

11. The method of claim 7 wherein the alpha-hydroxy acid is applied in combination with a second topical solution, cream or lotion.

12. The method of claim 7 wherein the alpha-hydroxy acid is applied in combination with an abrasive treatment.

13. The method of claim 12 wherein the abrasive treatment is micro-dermabrasion.
